(19) United States
(12) Patent Application Publication
(10) Pub. No.: US 2023/0074758 A1
(43) Pub. Date: Mar. 9, 2023

(54) METHOD AND DEVICE FOR TRANSCATHETER TREATMENT OF AN ASCENDING AORTA ANEURYSM

(71) Applicant: TT3A S.r.l., Milan (IT)

(72) Inventors: Pierfrancesco Veroux, Catania (IT); Federico De Marco, Milano (IT); Mehmet Hakan Apkinar, Istanbul (TR); Enrica Tamburino, S. Agata li Battiati (CT) (IT); Claudia Tamburino, S. Agata li Battiati (CT) (IT); Thomas NiBl, Mackenrode (DE)

(73) Assignee: TT3A S.r.l., Milan (IT)

(21) Appl. No.: 17/986,650

(22) Filed: Nov. 14, 2022

Related U.S. Application Data
(63) Continuation of application No. PCT/US21/31874, filed on May 11, 2021.
(60) Provisional application No. 63/023,485, filed on May 12, 2020.

(51) Int. Cl. A61F 2/07; A61F 2/24

(52) U.S. Cl. CPC A61F 2/07; A61F 2/24; A61F 2/95

(57) ABSTRACT

An endoluminal prosthesis includes a stent-graft and a temporary aortic valve, typically combined in an integrated assembly suitable for transfemoral or other endoluminal placement in a patient's ascending aorta, aortic root, and aortic valve. The stent-graft has a base end configured to be positioned into over the patient's aortic root and over the aortic annulus. The temporary aortic valve assembly is attached to the base end of the stent-graft and comprises a scaffold configured to be anchored in the patient's aortic annulus and valve leaflets configured to function temporarily after the endoluminal prosthesis has been implanted. At least one fenestration suitable for receiving a guidewire and/or a coronary stent graft is located near a junction between the base end of the stent graft and the temporary aortic valve, wherein said at least one fenestration is disposed on the endoluminal prosthesis to be aligned with one of the patient's coronary ostia after the endoluminal prosthesis has been implanted.

### CROSS-REFERENCE TO RELATED APPLICATIONS

This application is a continuation of PCT Application PCT/US2021/031874 (Attorney Docket No. 64101-703.601) filed May 11, 2021, which claims the benefit of U.S. Provisional No. 63/023,485 (Attorney Docket No. 64101-703.101), filed May 12, 2020, the entire content of which is incorporated herein by reference.

### BACKGROUND OF THE INVENTION

1. Field of the Invention. The present application relates generally to medical devices and methods, and more particularly to an implantable, temporary endoluminal prosthesis for the repair of aa aortic aneurysm and methods for its implantation and the implantation of a permanent prosthetic aortic valve.

Thoracic aortic diseases including aneurysms and dissections of the thoracic aorta are a major cause of morbidity and mortality. Aortic aneurysms can occur in any segment of the aorta, including the aortic root and/or the ascending aorta above the patient's aortic valve. Such aneurysms are progressive and are dangerous because they rarely produce symptoms before complications occur. If it is not identified and managed correctly, the outcome can be fatal at any age.

Aneurysms in the ascending aorta can be detected by conventional imaging modalities such as X-Ray, echocardiography, computed tomography, magnetic resonance imaging and the like. When detected and deemed suitable, treatment of aneurysms in the ascending aorta typically comprises an open surgical procedure, such as a Bentall procedure, where a composite graft is used to replace the patient's aortic valve, aortic root, and ascending aorta, with implantation of the coronary arteries into the graft. This operation is particularly useful to treat combined disease in both the aortic valve and the ascending aorta. Most patients undergoing a Bentall procedure will also receive a mechanical valve.

Endovascular repair of an aneurysm in the ascending aorta is complicated by the need for short grafts, which may be less stable during or after deployment. If a second stent-graft must be placed, there is the risk of the first stent-graft being displaced as the second device is advanced into position, a dangerous and difficult complication to resolve intra-operatively. Also, placement of a stiff delivery wire and the stent-graft delivery system in the left ventricle increases the risk for perforation or rupture of the left ventricle.

Gaia et al., JACC: Case Reports, February 2020, describe a case study of patient who had a pseudoaneurysm in the ascending aorta and a severely calcified porcelain aorta. After percutaneous treatment failed, an implant was custom-designed based on the patient's CT, including a balloon-expandable transcatheter proximal prosthesis connected to a self-expandable aortic stent. The procedure was referred to as the “Endo-Bentall” procedure as it mimicked the open surgical Bentall procedure described above.

While successful, the Endo-Bentall procedure suffers from certain short comings. It requires that the implant be custom designed and built, greatly increasing complexity and cost. Rapid pacing was required during implantation, and catheterization of the branch and coronary arteries proved difficult. Extracorporeal blood oxygenation (ECMO) was also required to support the patient.

U.S. Pat. No. 8,715,337 describes an implantable endoluminal prosthesis for permanently replacing a damaged aortic valve. The prosthesis includes a balloon-expandable stent, a tubular conduit that extends into the ascending aorta, and a self-expanding stent. The tubular conduit extends across the balloon-expandable stent. The tubular conduit includes an artificial valve. The self-expanding stent extends across the tubular conduit into the ascending aorta. The balloon-expandable stent, the tubular conduit, and the self-expanding stent are coupled to provide unidirectional flow of fluid into the aorta and further into the coronary arteries.

For these reasons, it would be desirable to provide improved devices and methods for treating disease in a patient's ascending aorta while facilitating prosthetic replacement of the patient's aortic valve. In particular, it would be desirable to advance a treatment implant using transfemoral approach, avoid the need for rapid pacing during implantation, avoid the need for ECMO to support coronary perfusion during implantation, rely on self-expansion of the implant rather than balloon-expansion, and achieve stable placement with little or no migration of the implant after implantation. It would be further desirable if the implant were compatible for use with other available prosthetic valves to enhance versatility and doctor choice. At least some of these objectives will be met by the inventions described and claimed hereinbelow.

2. Listing of the Background Art. Gaia et al., JACC: Case Reports, February 2020 and U.S. Pat. No. 8,715,337 have been discussed above. See also U.S. Pat. No. 8,940,040 and Clare et al., Curr Atheroscler Rep (2016) 18:60.

### SUMMARY OF THE INVENTION

In a first aspect, the present invention provides an endoluminal prosthesis comprising a stent-graft and a temporary aortic valve, typically combined in an integrated assembly suitable for transfemoral or other endoluminal placement in a patient's ascending aorta, aortic root, and aortic valve. The stent-graft has a base end configured to be positioned into over the patient's aortic root and over the aortic annulus. The temporary aortic valve assembly is attached to the base end of the stent-graft, and comprises a scaffold configured to be anchored in the patient's aortic annulus and valve leaflets configured to function temporarily after the endoluminal prosthesis has been implanted. At least one fenestration suitable for receiving a guidewire and/or a coronary stent graft is located near a junction between the base end of the stent graft and the temporary aortic valve, wherein said at least one fenestration is disposed on the endoluminal prosthesis to be aligned with one of the patient's coronary ostia after the endoluminal prosthesis has been implanted.

In some embodiments, the endoluminal prostheses of the present invention may further comprise at least one coronary stent-graft configured to be implanted through said at least one fenestration after the endoluminal prosthesis has been implanted. Preferably, such endoluminal prostheses will comprise two fenestrations disposed on the endoluminal prosthesis to be aligned with two of the patient's coronary ostia after the endoluminal prosthesis has been implanted and two coronary stent-grafts configured to be implanted through said two fenestrations after the endoluminal prosthesis has been implanted. After implanting, the coronary stent-grafts will usually form a bridge with the central or main stent-graft which both stabilizes the central or main stent-graft and inhibits blood leakage outside of the stent-graft assembly.

In some embodiments, wherein the temporary aortic valve assembly may include an anchor structure about its periphery, where the anchor structure is typically configured to anchor the temporary aortic valve assembly in the patient's native aortic leaflets. For example, the anchors structure may comprise a plurality of barbs distributed over a circumference thereof.

In some embodiments, the stent-graft may taper radially outwardly in a direction away from the base.

In some embodiments, the stent-graft may have a length which terminates before reaching the aortic side branch vessels.

In some embodiments, the scaffold of the temporary aortic valve assembly may have an hourglass shape.

In some embodiments, the leaflets may be formed from a polymeric material.

In a second aspect, the present invention provides a method for delivering a prosthetic heart valve to a patient. The endoluminal prosthesis comprises a stent-graft and a temporary aortic valve assembly including a scaffold and temporary valve leaflets, preferably having any of the design features described previously and elsewhere herein. The endoluminal prosthesis is implanted in the patient's beating heart with the stent graft located in the patient's ascending aorta and the temporary valve assembly located in the patient's native aortic valve. The temporary valve takes over the patient's aortic valve function and is configured to receive a permanently implanted prosthetic aortic valve expanded within the temporary valve leaflets of the scaffold.

In some embodiments of the methods of the present invention, the permanent prosthetic aortic valve is implanted within the temporary valve leaflets of the scaffold. The temporary valve leaflets are immobilized and no longer function but are left in place and help anchor the permanent prosthetic aortic valve with the scaffold.

In preferred embodiments, all implanting steps are performed endovascularly, more preferably being performed via transfemoral access.

In other preferred embodiments, the endoluminal prosthesis has at least one fenestration located near a junction between a base end of the stent graft and the temporary aortic valve, and implantation further comprises aligning the at least one fenestration with one of the patient's coronary ostia as the endoluminal prosthesis is being implanted. Even more preferably, aligning the at least one fenestration with one of the patient's coronary ostia comprises introducing the endoluminal prosthesis over a guidewire place over the patient's aortic arch and into the coronary artery. Typically, the endoluminal prosthesis is introduced over two guidewires placed over the patient's aortic arch and through two fenestrations located near the junction between the base end of the stent graft and the temporary aortic valve and into the coronary artery. Optionally, the endoluminal prosthesis is simultaneously advanced over a third guidewire located over the patient's aortic arch and through the patient's native valve leaflets as the endoluminal prosthesis is being advanced over the two guidewires located in the coronary ostia.

In many instances, implanting the endoluminal prosthesis in the patient's beating heart comprises releasing the endoluminal prosthesis from radial constraint to allow said endoluminal prosthesis to self-expand, and often implanting the endoluminal prosthesis comprises anchoring the endoluminal prosthesis with barbs located about a circumference thereof.

### BRIEF DESCRIPTION OF THE DRAWINGS

FIG. 1 illustrates an endoluminal prosthesis including a distal stent-valve assembly joined to a proximal stent-graft constructed in accordance with the principles of the present invention.

FIG. 2 is a cross-sectional view taken along line 2-2 of FIG. 1 showing placement of a temporary valve in the distal stent-valve assembly of the endoluminal prosthesis of the present invention.

FIG. 3 is a detailed view of section 3-3 of FIG. 1 showing fenestrations positioned in a distal end of the stent-graft of the endoluminal prosthesis of the present invention.

FIG. 4 is a schematic illustration of an endoluminal prosthesis of the present invention illustrating certain structural details of exemplary embodiments.

FIG. 5 illustrates a delivery catheter of the present invention configured to deliver the endoluminal prostheses to a patient's heart.

FIGS. 6A to 6C show guidewire placement and release of the endoluminal prosthesis from constraint on the delivery catheter of FIG. 5.

FIGS. 7A to 7F illustrate use of the delivery catheter of FIG. 5 for placement of the endoluminal prosthesis of FIG. 1 into a patient's aortic valve and subsequent delivery of a permanent prosthetic aortic valve.

### DETAILED DESCRIPTION OF THE INVENTION

Referring to FIG. 1, an endoluminal prothesis 100 constructed in accordance with the principles of the present invention comprises a stent-graft 102 and a scaffold-valve assembly 104. The stent-graft 102 includes both a membrane 112 and a supporting scaffold 110. The supporting scaffold 110 may comprise any conventional scaffold structure or stent used in cardiovascular applications. Thus, while illustrated as a series of separate serpentine rings which are distributed along the length of the stent-graft portion 102, the scaffold could be a helical structure, an open-cell scaffold, a closed-cell scaffold, a series of connected serpentine, zig-zag, or other rings, or any one of many other graft supporting structures well known in the art.

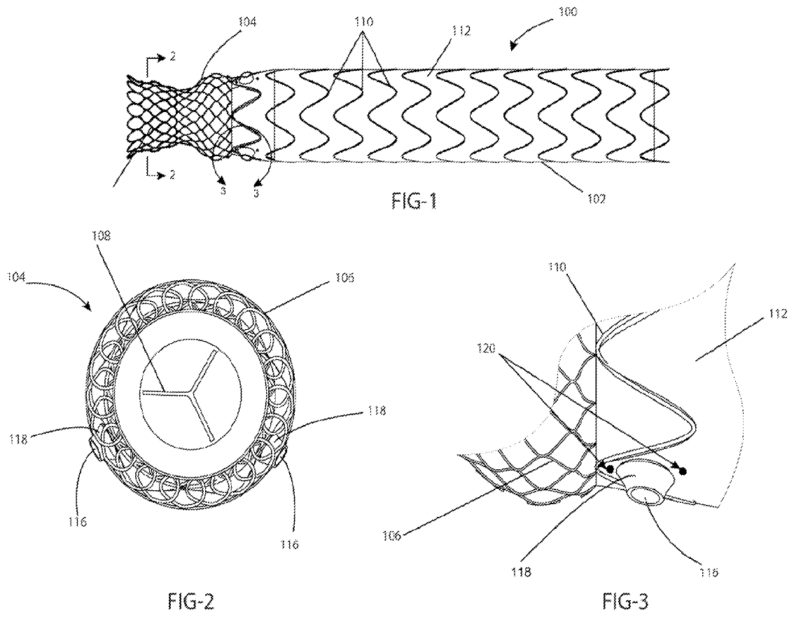

Referring now to both FIGS. 1 and 2, the scaffold-valve assembly 104 includes an external scaffold 106 and an internal temporary valve 108. The valve 108 is secured on an interior surface of the scaffold 106 at a location near the midpoint of the scaffold 106, and the scaffold 106 is typically formed with a narrow-diameter waist region, usually having an hourglass configuration, where the valve 108 is usually attached to an inner surface of the waist region of the scaffold.

Referring now to FIGS. 1 and 3, the stent-graft 102 will usually include one or more fenestrations 116, typically being formed within a conical port 118 formed in a wall or surface of the membrane 112.

The fenestrations 116 will typically be formed in an end of the stent-graft 102 immediately adjacent to the scaffold-valve assembly 104. As illustrated in FIG. 3, the conical portion 118 may be nested within the crown of a serpentine ring of the scaffold 110. There will usually be a pair of fenestrations 116, and their location will allow the endoluminal prothesis 100 to be delivered over a pair of laterally oriented guide wires in the coronary arteries to help guide placement of the endoluminal prothesis as will be described in more detail below. The guidewires may optionally be anchored with an anchoring means, such as a coiled tip, an expanded diameter, a balloon over the guidewire, or a percutaneous transluminal coronary angioplasty (PTCA) catheter inflated to keep the guidewires in position and anchored safely during subsequent advancement of the delivery system.

FIG. 3 further shows a pair of radiopaque markers 120 located adjacent the fenestrations 116 in the conical ports 118. The radiopaque markers 120 can be by the physician to locate the fenestrations adjacent the patient's coronary arteries as the endoluminal prothesis 100 is being delivered, as will be described in greater detail below.

The scaffolds of the stent-graft 102 and the scaffold-valve assembly 104 will typically be formed from a shape memory material, typically from a nickel-titanium alloy. The scaffold of the stent-graft 102 will usually be a bent wire structure, while the scaffold of the scaffold-valve assembly 104 will typically be patterned, e.g. laser cut, from a tube, although the latter might also be an expandable, metallic braided stent frame.

The graft (cover) of the stent-graft 102 may be any conventional graft material, such as polytetrafluoroethylene (PTFE), polyethylene terephthalate (PET), e.g. Dacron®, and the like.

The temporary valve may be formed from the same or similar material as the graft, e.g. as PTFE and PET, as well as from bioabsorbable polymeric materials as the temporary valve leaflets do not need to be persistent.

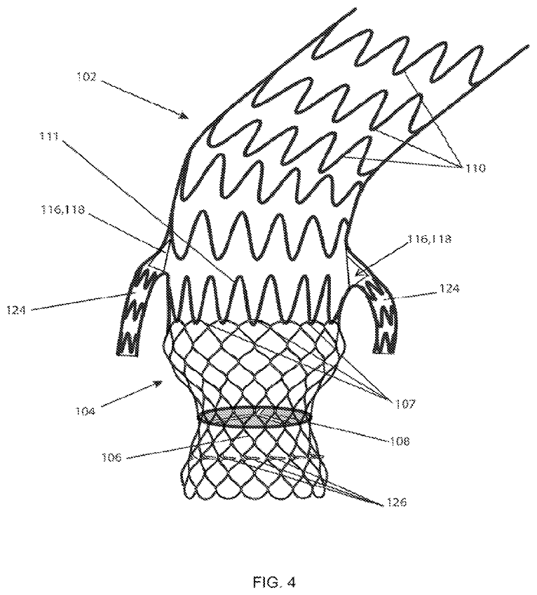

Referring now to FIG. 4, a schematic illustration of the endoluminal prothesis 100 shows a preferred connection between the scaffold 110 of the stent-graft 102 and the scaffold 106 of the scaffold-valve assembly 104. End loops of a terminal serpentine ring 111 of the scaffold 110 may be overlapped with terminal loops 107 of the scaffold 106 of the scaffold-valve assembly 106. As shown in FIG. 4, the scaffold 106 will typically be a closed-cell structure (usually patterned from a cylindrical metal sleeve) while the scaffold 110 of the stent-graft 102 will be formed from bent wires, allowing the wires to be passed through the closed cells of the scaffold 106. The bent wire scaffold 106 may be formed as independent serpentine or other rings or alternatively may be formed as a continuous helical serpentine structure.

FIG. 4 further shows another preferred feature of the present invention. A plurality of tissue-anchoring barbs 126 may be formed about a circumference of the scaffold 106 of the scaffold-valve assembly 104, where the barbs are intended to help stabilize the scaffold-valve assembly in tissue of the valve leaflets after the scaffold-valve assembly is expanded in the patient's aortic valve, as described in more detail below.

FIG. 4 still further illustrates the optional inclusion of a pair of coronary stent-grafts 124 which are typically formed separately from the stent-graft 102, allowing separate delivery after the stent-graft 102 has been deployed, as described in greater detail below. The coronary stent-grafts 124 can help treat disease in the coronary root as well as enhance stabilization of the stent-graft 102 in the ascending aorta.

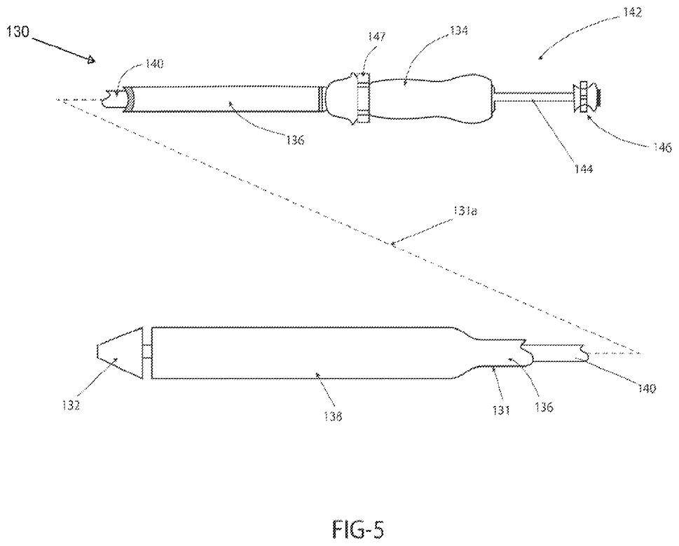

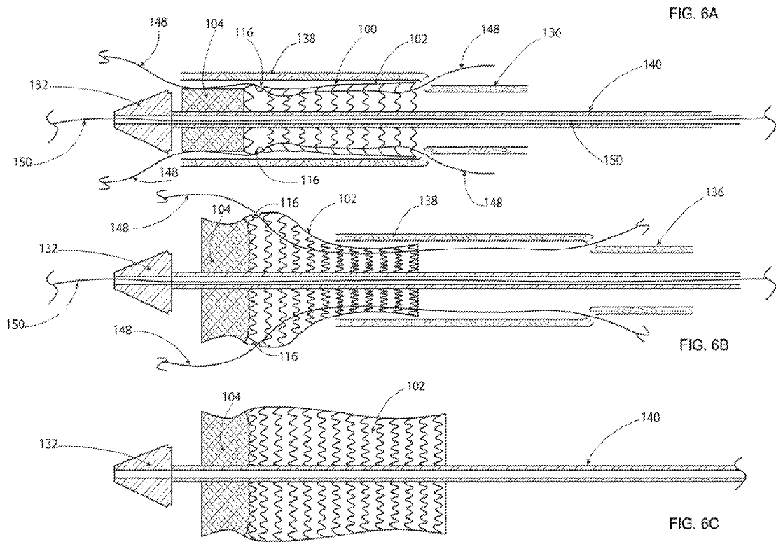

Referring now to FIG. 5, a delivery catheter 130 useful for delivering the endoluminal protheses 100 of the present invention to a patient's ascending aorta and aortic valve will be described. The delivery catheter 130 comprises a catheter body 131 extending along a longitudinal axis 131a from an atraumatic distal tip 132 and a proximal handle 134. The catheter body 131 includes an inner tubular shaft 140 and an outer tubular member 136. The outer tubular member 136 includes a constraining capsule 138 at its distal end. The outer tubular member 136 is configured to be translated between a distal-most position over the inner shaft 140, as shown in FIG. 5, and a proximally retracted configuration relative to the shaft as described with reference to FIG. 6A through 6C. Retraction of the outer tubular member 136 and constraining capsule 138 relative to the inner shaft 140 (which carries the endoluminal prothesis 100) is effected using any conventional pullback assembly. An exemplary pullback assembly 142 includes a screw drive 144 and a rotating lock member 146. The user may hold the handle 134 in one hand while rotating the handle lock mechanism 146 to pull back the handle 134 and outer tubular member 136 relative to the inner shaft 140. A steering knob 147 is also provided which is attached to two pull wires (not illustrated) which steer the distal end of the catheter in a conventional manner. While a specific handle configuration is illustrated, it will be appreciated that a variety of stent and stent-graft deployment catheters and handles including both steering mechanisms and sheath pull back assemblies are well known in the art and could be employed in the present invention.

Referring now to FIG. 6A through 6C, release of the endoluminal prothesis 100 from the delivery catheter 130 will be described in more detail. Initially, the stent-graft 102 and stent-valve assembly 104 are constrained within the constraining capsule 138 of the outer tubular member 136 while the outer tubular member is in its distal-most configuration, as shown in FIG. 6A. When in this “delivery” configuration, a pair of lateral or side guide wires 148 (typically 0.014 in.) may be positioned through a gap between the atraumatic distal tip 132 and the distal end of the constraining capsule 138. The guide wires 148 pass beneath an interior surface of the constraining capsule 138 and over an exterior surface of the scaffold-valve assembly 104 and then through the fenestrations 116 and into an interior of the stent-graft 102 of the endoluminal prosthesis 100.

After passing through the stent-graft 102, the lateral guide wires 138 pass outwardly through a rear surface of the constraining capsule 138. A main or central guide wire 150 (typically 0.035 in.) will typically be positioned through a central lumen of the inner shaft 140.

As shown in FIG. 6B, the outer tubular member 136 may be retracted (using the pullback assemblies described previously) allowing the endoluminal prosthesis 100 to self-expand beginning at its distal end. Thus, the scaffold-valve assembly 104 will radially expand before the stent-graft 102 expands. As the constraining capsule 138 is retracted and endoluminal prosthesis 100 radially expands, distal portions of the lateral guidewires 148 continue to pass outwardly through the fenestrations 116 while they are released from the constraining capsule 138. Intermediate portions of the lateral guidewires 148 remain located within the stent-graft 102, and proximal portions will be realsed after the outer housing member 136 is removed.

As shown in FIG. 6C, the endoluminal prosthesis 100 including both the stent-graft 102 and scaffold-valve assembly 104 will fully expand after the constraining capsule 138 is drawn fully proximally freeing the endoluminal prosthesis 100 from radial constraint.

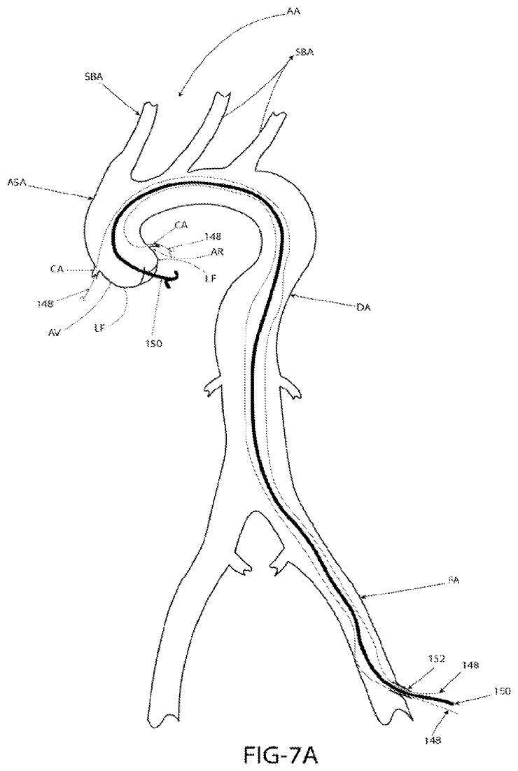

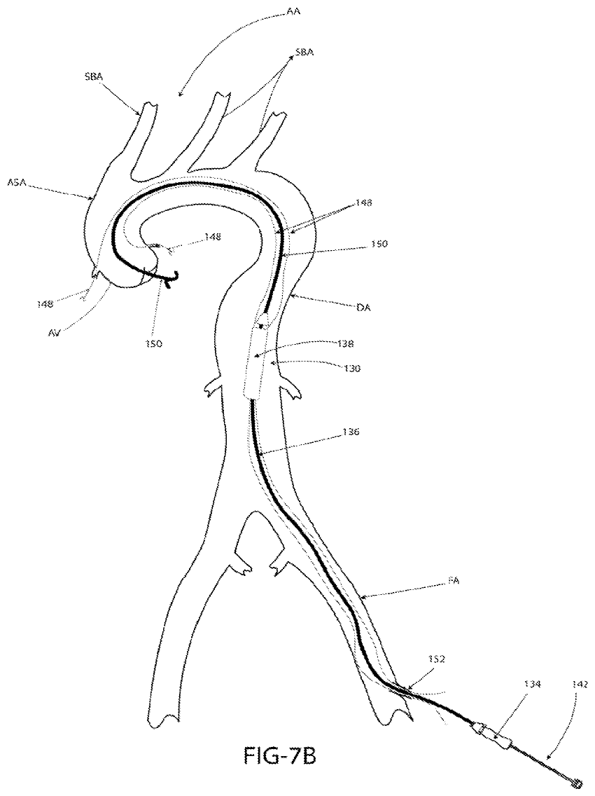

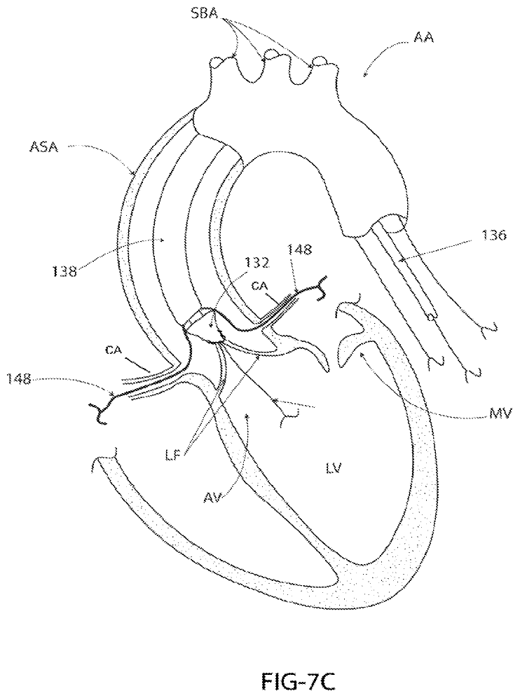

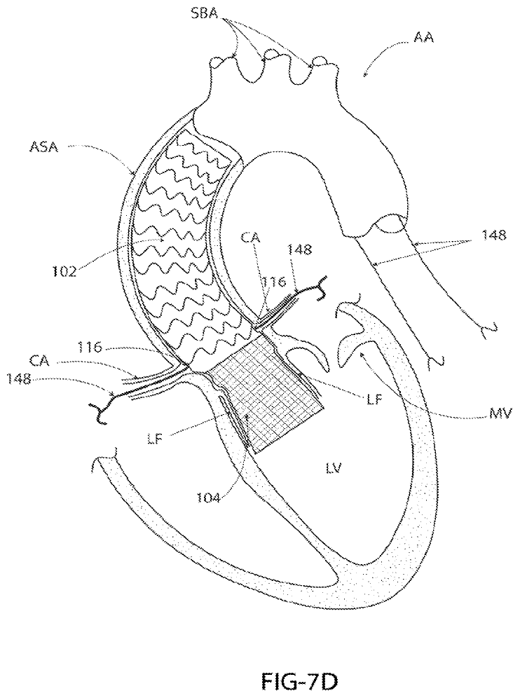

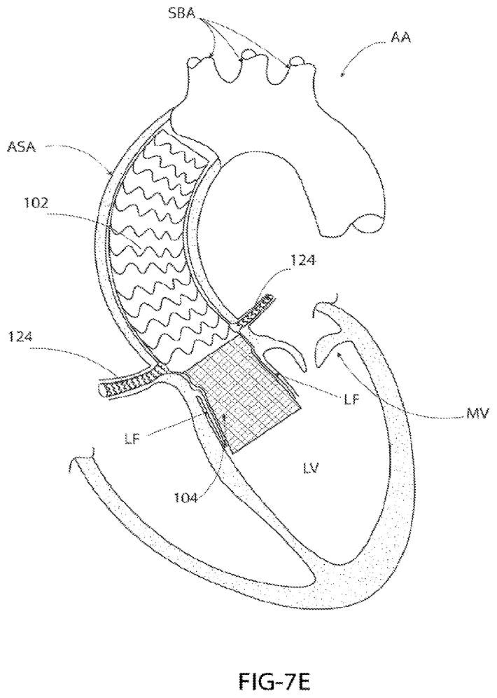

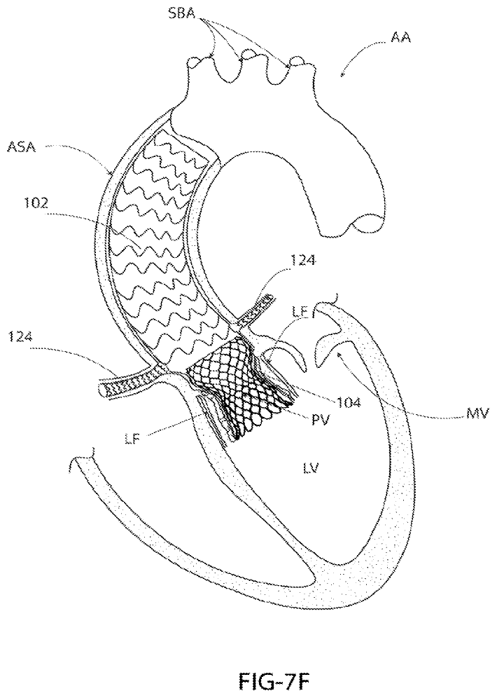

Referring now to FIGS. 7A through 7F, an exemplary protocol for delivering the endoluminal prosthesis 100 to a patient's ascending aorta ASA and aortic valve AV will be described. An access sheath 152 is introduced into the patient's femoral artery FA in the patient's groin, in a conventional manner. Lateral guidewires 148 and 150 are then passed through the access sheath 152 and over the patient's aortic arch AA, with one lateral guidewire 148 passing through each of the coronary arteries CA adjacent the patient's aortic root AR. The main guidewire 150 is introduced through the access sheath 152, over the aortic arch AA and through the leaflets LF of the aortic valve AV.

After the guidewires 148 and 150 have been placed, the delivery catheter 130 will be introduced over the guidewires toward the patient's aortic arch AA, as shown in FIG. 7B. As can also be seen in FIG. 7B, the delivery catheter 130 is advanced with the main guidewire 150 passing through the lumen of the inner shaft 140 while the lateral guidewires 148 pass beneath the constraining capsule 138 and inwardly through the fenestrations 116, as seen in FIGS. 6A to 6C.

As shown in FIG. 7C, as the atraumatic tip 132 of the delivery catheter 130 approaches the aortic valve AV, the lateral guidewires 148 which are anchored in the coronary arteries CA, will begin to slow the advancement of the catheter 130 to help the physician properly locate the catheter properly relative to the aortic valve AV. The main guidewire 150 will assure that the catheter remains positioned to pass through the native leaflets LF. Placement of the endoluminal prosthesis 100 will be performed under fluoroscopic imaging, with tactile feedback from the lateral guidewires providing supplemental positioning information.

After fully advancing the distal portion of the delivery catheter 130, the constraining capsule 138 will be retracted allowing both the scaffold-valve assembly 104 and the stent-graft 102 to expand in situ in the aortic valve AV and ascending aorta ASA, respectively, as shown in FIG. 7D. The lateral guidewires 148 assure that the fenestrations 116 are properly positioned adjacent the coronary arteries CA.

As soon as the endoluminal prosthesis 100 is positioned as shown in FIG. 7D, the temporary valve 108 (FIG. 2) can begin to function as the native valve leaflets LF are opened and immobilized in the left ventricle LV. At this point, the main guidewire 150 can be withdrawn and each of the lateral guidewires 148 can be used to separately deliver a coronary stent graft 124 to each of the coronary arteries CA, as shown in FIG. 7E.

After delivery of the coronary stent grafts 124, a permanent prosthetic valve PV can be delivered to supplant the temporary valve 108, as shown in FIG. 7F. The prosthetic valve can be any one of presently available valves which are intended to be delivered within external supporting stents, where the scaffold-valve assembly 104 of the present and mentioned will act as the external supporting stent.

### Example

An endoprosthesis 100 as illustrated in FIG. 1 and delivery catheter 130 as illustrated in FIG. 5 are provided. An access sheath 152 is placed through the patient's groin into the femoral artery using the Seldinger technique, and a 0.035 in. guidewire is advanced into the left ventricle and two 0.014 in. coronary guidewires are placed in the right and left coronaries. The coronary arteries are optionally anchored at their distal ends in the coronary arteries. The 0.035 in. guidewire is loaded into the distal end of the main guidewire lumen, and the two 0.014 in. coronary guidewires are loaded the fenestrations 116 in the distal end of the stent-graft 102 while the constraining capsule 138 is partially retracted. The constraining capsule is then advanced distally back over the 0.014 in. wires which exit the delivery catheter 130 through a gap between the distal end of the constraining capsule 138 and the proximal end of the atraumatic tip 132.

The delivery catheter 130 is loaded to a loader and flushed with a saline solution to remove residual air bubbles which might cause air-embolization. The delivery catheter 130 is advanced from the groin to the aortic valve under fluoroscopic imaging. When the distal end of the delivery catheter 130 reaches the coronary arteries, the constraining capsule 138 is partially retracted to allow the scaffold 104 of the scaffold-valve assembly 106 to partially expand and free the 0.014 in. guidewires from the constraining capsule 138 (as shown in FIG. 6B).

The delivery catheter 130 is then manipulated to align the fenestrations 116 with the coronary ostia. Alignment can be confirmed based on observing the radiopaque markers 120 on the fluoroscopic image and tactile feedback of the alignment of the 0.014 in. guidewires with the coronary ostia.

After placement of the endoluminal prosthesis 100 and removal of the delivery catheter 130, the barbs 126 hold the endoprosthesis in place against the native lave leaflets, the temporary valve 108 begins functioning, and blood starts to enter the coronary arteries. The coronary stent-grafts 124 may then be delivered over the 0.014 in. guidewires through the coronary access fenestrations 116 on the stent-graft 102, forming a bridge between the stent-graft and the coronary arteries in both coronary ostia to seal the graft area from any potential endo leak. At this stage a commercially available prosthetic aortic valve (TAVR) intended for permanent implantation may advanced and implanted inside the temporary valve.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An endoluminal prosthesis comprising:
a stent-graft configured for endoluminal placement in a patient's ascending aorta, said stent graft having a base end configured to be positioned over the patient's aortic annulus;
a temporary aortic valve assembly attached to the base end of the stent-graft, said temporary aortic valve assembly having scaffold configured to be anchored in the patient's aortic annulus and valve leaflets configured to function temporarily after the endoluminal prosthesis has been implanted; and
at least one fenestration located near a junction between the base end of the stent graft and the temporary aortic valve, wherein said at least one fenestration is disposed on the endoluminal prosthesis to be aligned with one of the patient's coronary ostia after the endoluminal prosthesis has been implanted.

2. The endoluminal prosthesis of claim 1, further comprising at least one coronary stent-graft configured to be implanted through said at least one fenestration after the endoluminal prosthesis has been implanted.

3. The endoluminal prosthesis of claim 2, comprising two fenestrations disposed on the endoluminal prosthesis to be aligned with two of the patient's coronary ostia after the endoluminal prosthesis has been implanted and two coronary stent-grafts configured to be implanted through said two fenestration after the endoluminal prosthesis has been implanted.

4. The endoluminal prosthesis of any one of claims 1 to 3, wherein the temporary aortic valve assembly includes an anchor structure about its periphery, wherein said anchor structure configured to anchor the temporary aortic valve assembly in the patient's native aortic leaflets.

5. The endoluminal prosthesis of claim 4, wherein the anchors structure comprises a plurality of barbs distributed over a circumference thereof.

6. The endoluminal prosthesis of claim 1, wherein the stent-graft tapers radially outwardly in a direction away from the base.

7. The endoluminal prosthesis of claim 6, wherein the stent-graft has a length which terminates before reaching the aortic side branch vessels.

8. The endoluminal prosthesis of claim 1, wherein the scaffold of the temporary aortic valve assembly has an hourglass shape.

9. The endoluminal prosthesis of claim 1, wherein leaflets are formed from a polymeric material.

10. The endoluminal prosthesis of claim 2, wherein the fenestrations are formed in a conical port structure formed in the stent-graft.

11. The endoluminal prosthesis of claim 10, wherein the conical port structure is formed in a graft wall of the stent-graft.

12. The endoluminal prosthesis of claim 11, wherein the conical port structure is configured to form a bridging structure with the coronary stent-graft.

13. A method for delivering a prosthetic heart valve to a patient, said method comprising:
providing an endoluminal prosthesis comprising a stent-graft and a temporary aortic valve assembly including a scaffold and temporary valve leaflets; and
implanting the endoluminal prosthesis in the patient's beating heart with the stent graft located in the patient's ascending aorta and the temporary valve assembly located in the patient's native aortic valve, wherein the temporary valve takes over the patient's aortic valve function;
wherein the temporary aortic valve assembly is configured to receive a permanently implanted prosthetic aortic valve expanded within the temporary valve leaflets of the scaffold.

14. The method of claim 13, further comprising implanting the permanent prosthetic aortic valve within the temporary valve leaflets of the scaffold.

15. The method of claim 13, wherein all implanting steps are performed endovascularly.

16. The method of claim 15, wherein all implanting steps are performed transfemorally.

17. The method of claim 13, wherein the endoluminal prosthesis has at least one fenestration located near a junction between a base end of the stent graft and the temporary aortic valve, further comprising aligning the at least one fenestration with one of the patient's coronary ostia as the endoluminal prosthesis is being implanted.

18. The method of claim 17, wherein aligning the at least one fenestration with one of the patient's coronary ostia comprises introducing the endoluminal prosthesis over a guidewire placed over the patient's aortic arch and into the coronary artery.

19. The method of claim 18, wherein aligning the at least one fenestration with one of the patient's coronary ostia comprises introducing the endoluminal prosthesis over two guidewires placed over the patient's aortic arch and through two fenestrations located near the junction between the base end of the stent graft and the temporary aortic valve and into the coronary artery.

20. The method of claim 18, wherein the endoluminal prosthesis is simultaneously advanced over a third guidewire located over the patient's aortic arch and through the patient's native valve leaflets as the endoluminal prosthesis is being advanced over the two guidewires located in the coronary ostia.

21. The method of claim 13, wherein implanting the endoluminal prosthesis in the patient's beating heart comprises releasing the endoluminal prosthesis from radial constraint to allow said endoluminal prosthesis to self-expand.

22. The method of claim 13, wherein implanting the endoluminal prosthesis comprises anchoring the endoluminal prosthesis with barbs located about a circumference thereof.

23. The method of claim 18, further comprising implanting at least one coronary stent-graft through the fenestration and within the coronary artery.

24. The method of claim 23, wherein implanting the at least one coronary stent-graft comprises introducing the coronary stent-graft over the at least one guidewire.

25. The method of claim 24, wherein the at least one coronary stent-graft is implanted to bridge into the stent-graft.
